(19) United States
(12) Patent Application Publication
(10) Pub. No.: US 2021/0038527 A1
(43) Pub. Date: Feb. 11, 2021

(54) Capsule for Storing Lubricant

(71) Applicant: Innovemed Bio-Technology Co., Ltd., Taipei City (TW)

(72) Inventor: Yun-Wen Lee, Taipei City (TW)

(73) Assignee: Innovemed Bio-Technology Co., Ltd.

(21) Appl. No.: 16/731,138

(22) Filed: Dec. 31, 2019

(30) Foreign Application Priority Data
Aug. 8, 2019 (TW) 108128341

(51) Int. Cl. A61K 9/48

(52) U.S. Cl. CPC A61K 9/4866

(57) ABSTRACT

A capsule for storing lubricant is provided. The capsule includes a capsule shell. The capsule shell is filled with the lubricant. The ratio of raw materials of the capsule shell is gelatin:glycerin:water:humectants:thickener=53-74%:18-42%:6-16%:10-40%:2-20%. The ratio of raw materials of the lubricant is Cyclopentasiloxane:Dimethiconol:Polydimethylsiloxane=42-76%:18-42%:6-16%. The capsule shell has a pH range of dissolution, a temperature range of dissolution and a humidity range of dissolution.

### CROSS-REFERENCE TO RELATED APPLICATION

This application claims priority from Taiwan Patent Application No. 108128341, filed on Aug. 8, 2019, in the Taiwan Intellectual Property Office, the content of which is hereby incorporated by reference in its entirety for all purposes.

### BACKGROUND OF THE INVENTION

### 1. Field of the Invention

The present invention relates to a capsule, and especially relates to a capsule for storing lubricant applicable in sexual activities and medical treatment.

### 2. Description of the Related Art

In general, lubricant for skin of human bodies is commonly stored in jars, bottles or other kinds of containers. People get used to pour or squeeze the lubricant stored in the containers when needing the lubricant. However, some issues related with the ways of the storage of the lubricant are raised as followings:

1. Repeatedly dispensing the lubricant with hands not thoroughly cleaned might increase the risk of infection of bacteria or viruses, and thereby leading to an issue of health and safety.

2. Since the lubricant is stored in a container, it is not convenient enough for carrying. Besides, the container may occupy a substantial space and therefore it might not be elegant while using the lubricant. In addition, in view of the aforementioned step for dispensing the lubricant, the user experience might be more unpreferable.

One of the related disclosures is Taiwan Patent M544316. The disclosure relates to a container structure and a stress-controlled container for aqueous compounds, wherein a bottle is included. The bottle has a bullet type accommodating space therein. The bullet type accommodating space is configured to contain an aqueous compound including aloe extract and hyaluronic acid. When the bottle is squeezed, the aqueous compound comes out from the bottle.

Taiwan Patent M526400 relates a capsule, which may disintegrate in the stomach. The capsule includes a capsule shell and natural and active peptide powder, wherein the capsule shell is capable of being dissolved in the stomach, and the natural and active peptide powder is filled in the capsule.

China Patent Application CN103598971A relates an electric sex toy having a function of lubrication. The sex toy includes a driving portion, a lubricating portion, a control portion and an imitative organ, wherein the driving portion may drive the imitative organ to move as naturally as human bodies. Further, the lubricating portion may effectively store lubricant. The lubricant is then pumped from the lubricating portion by a pump and guided into the imitative organ via a leveling pipe. A human body is hence lubricated when wearing the imitative organ.

China Patent Application CN104352679A relates to a capsule for lubricating bowels and inducing bowel movements. The capsule includes the capsule shell and extract of some Chinese herbal medicines filled therein. This prior disclosure further disclosed that the raw materials of the extract of the Chinese herbal medicines are aloe extract, seeds of Urtica macrorrhiza Hand.-Mazz., Semen Raphani, fetid Cassia seed, and apricot kernel, and the ratio of raw materials of the capsule shell is gelatin:glycerin:alcohol:pure water=3-5%:1%-3%:5-8%:84-91%. The capsule for lubricating bowels and inducing bowel movements is lubricative, and hence may lubricate the inner wall of the rectums. Further, the capsule is soluble in water.

U.S. Pat. No. 6,749,557B2 relates to a physical therapy apparatus, which provides the function of lubrication by the lubricant stored therein. The apparatus includes a cavity and a connection mechanism, wherein the cavity is used to load the lubricant and the connection mechanism is used to connect the cavity to the physical therapy apparatus. The physical therapy apparatus includes a surface structure for contacting the outer surface and the inner surface of the user, wherein the surface structure includes one or more holes. The user may control the connection mechanism to receive the lubricant of the cavity.

U.S. Pat. No. 7,858,119B1 relates a slow released medicine, which is stored in a polymer film and gradually releases the active drug material in a stomach, bowels and blood for 12-24 hours. The slow released medicine includes a shell, wherein the shell is a polymer film of a single layer or multiple layers and only soluble for a pH value higher than pH=5. The prior disclosure further disclosed that the shell includes polyethylene glycol plasticizer of 0.5-30% and polymer of 5-55%.

However, the above-mentioned disclosures are all silent about how to prevent the capsule shell from being corroded by the lubricant stored therein under the room temperature.

### SUMMARY OF THE INVENTION

The purpose of the present application is to provide a capsule for storing lubricant capable of solving the above-mentioned issues in the prior art.

A capsule for storing lubricant is provided. The capsule is used for sex and medical treatment. The capsule includes a capsule shell. The capsule shell is filled with the lubricant. The ratio of raw materials of the capsule shell is gelatin:glycerin:water:humectants:thickener=53-74%:18-42%:6-16%:10-40%:2-20%. The ratio of raw materials of the lubricant is Cyclopentasiloxane:Dimethiconol:Polydimethylsiloxane=42-76%:18-42%:6-16%. The capsule shell has a pH range of dissolution, a temperature range of dissolution and a humidity range of dissolution.

Preferably, the capsule shell may be a structure of a single layer.

Preferably, the thickness of the capsule shell may be 0.28-1.4 mm.

Preferably, the capsule shell may dissolve in a pre-determined period when the surroundings have a pH value in the pH range of dissolution, a temperature in the temperature range of dissolution, and humidity in the humidity range of dissolution.

Preferably, the pH range of dissolution may be 3.4-5.

Preferably, the temperature range of dissolution may be 30° C.-40° C.

Preferably, the humidity range of dissolution may be RH60-RH80.

The capsule shell of the capsule of the present invention for storing lubricant dissolves when the pH value, temperature and humidity of the surroundings are respectively in the pH range of dissolution, the temperature range of dissolution, and the humidity range of dissolution. Consequently, the lubricant stored in the capsule may be applied to the human bodies and provide the function of lubrication.

### BRIEF DESCRIPTION OF THE DRAWINGS

FIG. 1 depicts the capsule for storing lubricant according to an embodiment of the present application.

### DESCRIPTION OF THE PREFERRED EMBODIMENTS

In order to sufficiently understand the configuration and effect of the present inventive concepts, some example embodiments of the present inventive concepts will be described with reference to the accompanying drawings. It should be noted, however, that the present inventive concepts are not limited to the following exemplary embodiments, and may be implemented in various forms. Rather, the exemplary embodiments are provided only to disclose the present inventive concepts and let those skilled in the art fully know the scope of the present inventive concepts.

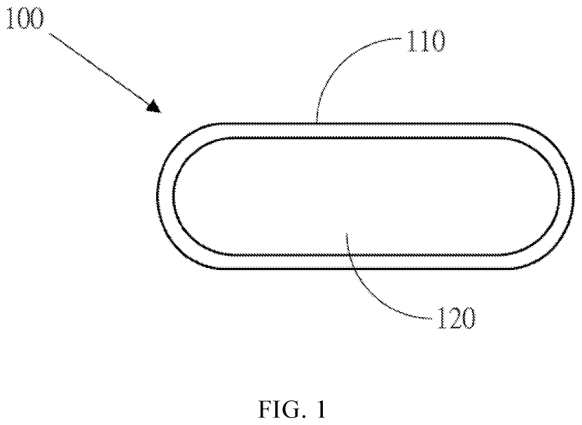

Refer to FIG. 1, which depicts the capsule for storing lubricant according to an embodiment of the present application. As shown in FIG. 1, the capsule 100 for storing lubricant is applied in sexual activities and medical treatment. The capsule 100 includes the capsule shell 110 and the lubricant 120 stored in the capsule shell 110. The ratio of raw materials of the capsule shell 110 is gelatin:glycerin:water:humectants:thickener=53-74%:18-42%:6-16%:10-40%:2-20%. The ratio of raw materials of the lubricant 120 is Cyclopentasiloxane:Dimethiconol:Polydimethylsiloxane=42-76%:18-42%:6-16%. The capsule shell 110 has a pH range of dissolution, a temperature range of dissolution and a humidity range of dissolution.

When actually used, the capsule 100 for storing lubricant is placed on a specific portion of the human body. When the pH value, temperature and humidity of the surroundings are respectively in the pH range of dissolution, the temperature range of dissolution, and the humidity range of dissolution, the capsule shell 110 of the capsule of the present invention for storing lubricant starts to dissolve. The lubricant 120 stored in the capsule shell 110 would be released from the capsule shell 110 and contacts the human body.

It is worthy to note that the temperature range of dissolution and the humidity range of dissolution depend on the ratio of raw materials of the capsule shell 110. Because of the proper ratio of raw materials of the capsule shell 110, the capsule shell may dissolve when being applied to the human body and may be properly preserved and kept stably in the preservation environment rather than dissolve before being used.

Further, the lubricant 120 may also be preserved and kept stably in the capsule shell 110 due to the proper ratio of raw materials of the lubricant 120. Since the lubricant includes water and the included water having a higher ratio may soften the capsule shell 110, the ratio of the included water is kept as low as possible without diminishing the lubrication effect and function of the lubricant 120.

Further, the capsule 110 may be a structure of a single layer, wherein the thickness of the capsule shell 110 may be 0.28-1.4 mm.

The capsule shell 110 may dissolve in a pre-determined period when the surroundings have a pH value in the pH range of dissolution, a temperature in the temperature range of dissolution, and humidity in the humidity range of dissolution.

Further, the pH range of dissolution may be 3.4-5, the temperature range of dissolution may be 30° C.-40° C., and the humidity range of dissolution may be RH60-RH80.

Please refer to Table 1 shown below, which shows the experiment data of the capsule for storing lubricant of the present invention, wherein the conditions and method of the experiment are:

Dissolution Conditions:
Solvent: buffer solution of 50 mM (millimole) phosphate; pH 4.0; 500 ml
Temperature: 37° C.
Device II (paddle): 50 rpm

Experiment Method:
applying a dissolution system for the dissolution test (n=3) of the capsule for storing lubricant of the present invention and photographing for recording the required time for the capsule shell to rupture

TABLE 1

| Item (n) | Sample | Required time for rupturing |
|---|---|---|
| 1 | Capsule (thickness of | 19 minutes and 58 seconds |
| | 5.2-9 mm) | 25 minutes 31 seconds |
| | | 28 minutes 10 seconds |
| 2 | Capsule (thickness of | 17 minutes 7 seconds |
| | 2.2-4.6 mm) | 18 minutes 38 seconds |
| | | 19 minutes 10 seconds |
| 3 | Capsule (thickness of | 9 minutes 40 seconds |
| | 0.28-1.4 mm) | 12 minutes 25 seconds |
| | | 13 minutes 20 seconds |

The capsule shell of the capsule of the present invention for storing lubricant dissolves when the pH value, temperature and humidity of the surroundings are respectively in the pH range of dissolution, the temperature range of dissolution, and the humidity range of dissolution. Consequently, the lubricant stored in the capsule may be applied to the human bodies and provide the function of lubrication.

The above description is merely illustrative and not restrictive. Any equivalent modification or change without departing from the spirit and scope of the present disclosure should be included in the appended claims.

What is claimed is:

1. A capsule for storing lubricant applicable in sexual activities and medical treatment, and comprising:
a capsule shell; and
a lubricant;
wherein the capsule shell is filled with the lubricant, a ratio of raw materials of the capsule shell is gelatin:glycerin:water:humectants:thickener=53-74%:18-42%:6-16%:10-40%:2-20%, a ratio of raw materials of the lubricant is Cyclopentasiloxane:Dimethiconol:Polydimethylsiloxane=42-76%:18-42%:6-16%, the capsule shell has a pH range of dissolution, a temperature range of dissolution and a humidity range of dissolution, and the capsule shell dissolves when surroundings of the capsule shell have a pH value in the pH range of dissolution, a temperature in the temperature range of dissolution, and humidity in the humidity range of dissolution.

2. The capsule for storing lubricant of claim 1, wherein the capsule shell has a single layer in structure.

3. The capsule for storing lubricant of claim 1, wherein a thickness of the capsule shell is 0.28-1.4 mm.

4. The capsule for storing lubricant of claim 1, wherein the capsule shell dissolves in a pre-determined period when the surroundings have a pH value in the pH range of dissolution, a temperature in the temperature range of dissolution, and humidity in the humidity range of dissolution.

5. The capsule for storing lubricant of claim 1, wherein the pH range of dissolution is 3.4-5.

6. The capsule for storing lubricant of claim 1, wherein the temperature range of dissolution is 30° C.-40° C.

7. The capsule for storing lubricant of claim 1, wherein the humidity range of dissolution is RH60-RH80.
